(19) United States
(12) Patent Application Publication
(10) Pub. No.: US 2022/0125343 A1
(43) Pub. Date: Apr. 28, 2022

(54) SYSTEMS AND METHODS FOR A DIGITAL OTOACOUSTIC INTEGRATED OTOSCOPE

(71) Applicant: Vestigo Research LLC, Ketchum, ID (US)

(72) Inventors: Michael MCGINNIS, Duvall, WA (US); Harold Edward AYLIFFE, Ketchum, ID (US)

(73) Assignee: Vestigo Research LLC, Ketchum, ID (US)

(21) Appl. No.: 17/553,668

(22) Filed: Dec. 16, 2021

Related U.S. Application Data
(60) Provisional application No. 63/093,017, filed on Oct. 16, 2020.

(51) Int. Cl. A61B 5/12; H04R 1/02; H04R 1/08; A61B 1/227; A61B 1/05; A61B 1/00; G01S 17/88; G01J 5/00

(52) U.S. Cl. CPC A61B 5/126; H04R 1/02; H04R 1/08; A61B 1/2275; G01J 2005/0077; A61B 1/00172; G01S 17/88; G01J 5/0025; A61B 1/05

(57) ABSTRACT

An otoscope includes at least one speaker and two microphones. The speaker is configured to generate a stimulus pressure wave and direct a planarized pressure wave toward an ear canal of a patient when the otoscope is at least partially inserted into the ear canal of the patient. The microphones are configured to record the forward stimulus pressure wave as it travels toward the TM and the reverse response pressure wave of the patient as it travels toward the otoscope. The quantitative relationship between the stimulus and the response is the diagnostic measure. The microphones are configured to record the pressure wave, the otoscope is configured to analyze the pressure wave, and the otoscope is configured to display results to a medical professional or user. The speaker and microphones are positioned outside the ear canal of the patient when the otoscope is at least partially inserted into the ear canal.

### CROSS-REFERENCE TO RELATED APPLICATION

This application is a U.S. Non-Provisional application claiming the benefit of and priority to U.S. Provisional Application No. 63/093,017, filed Oct. 16, 2020, entitled DIGITAL OTOACOUSTIC INTEGRATED OTOSCOPE, the disclosure of which is incorporated herein by reference in its entirety.

### TECHNICAL FIELD

The present disclosure relates generally to otolaryngology and audiological instruments, and more particularly to a digital otoscope with integrated acoustic reflectance and otoacoustic emission measurements.

### BACKGROUND

Previous devices used to measure acoustic reflectance and emission properties of the ear during hearing exams are often considered to be invasive and poorly integrated. In some cases, they may even be cumbersome to use, expensive, and have narrow frequency bandwidth, limiting their utility. Current wideband acoustic immittance (WAI) measurements require use of a calibrated acoustic driver sealed in the ear canal. Because of this, currently used systems that conduct WAI measurements require a trained professional to use, are expensive and often uncomfortable for the patient. A device that is capable of making WAI and otoacoustic emission (OAE) measurements without requiring repeated calibrations or sealing devices in the ear canal is needed.

### SUMMARY

One aspect of the present disclosure is directed to an otoscope including at least one speaker and at least two microphones. The at least one speaker is configured to generate a pressure wave and to direct the pressure wave toward the tympanic membrane (TM) of a patient when the otoscope is at least partially inserted into the ear canal of the patient. The at least two microphones are configured to receive the pressure wave while sound is traveling toward the TM and after the sound wave has been partially reflected at the TM of the patient. The at least two microphones are configured to record the pressure wave, the otoscope is configured to analyze the pressure wave, and the otoscope is configured to display the results to a user such as an audiologist. In some instances, the device of the present disclosure may be used by a nurse and/or technician due to its simplified nature over devices previously used. The at least one speaker and the at least two microphones are positioned outside the ear canal of the patient when the otoscope is at least partially inserted into the ear canal of the patient.

Another aspect of the present disclosure relates to a method of examining an ear canal and the TM of a patient using an otoacoustic integrated otoscope. The otoscope includes at least one speaker and at least two microphones. The method includes at least partially inserting the otoscope into the ear canal of the patient. The at least one speaker and the at least two microphones are positioned outside the ear canal of the patient when the otoscope is at least partially inserted into the ear canal of the patient. The method also includes generating a pressure wave using the at least one speaker. The method further includes directing the pressure wave into the ear canal of the patient. The method also includes bouncing the pressure wave off of the tympanic membrane. The method further includes recording the pressure wave using the at least two microphones. The method analyzes the recorded pressure to separately quantify the sound wave exciting the ear, the sound wave reflected from the ear and the sound wave emitted from the ear, and displaying the results to a medical professional or user.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 illustrates a perspective view of an example of an otoscope for conducting hearing tests in accordance with the principals of the present disclosure.

FIG. 2 illustrates a front view of the exemplary otoscope shown in FIG. 1 in accordance with the present disclosure.

FIG. 3 illustrates a side view of the exemplary otoscope shown in FIG. 1 in accordance with the present disclosure.

FIG. 4 illustrates a sectional side view of the exemplary otoscope shown in FIG. 1 in accordance with the present disclosure.

FIG. 5 illustrates an example of a method of examining an ear with the exemplary otoscope shown in FIGS. 1-4 in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

This description provides examples, and is not intended to limit the scope, applicability or configuration of the invention. Rather, the ensuing description will provide those skilled in the art with an enabling description for implementing embodiments of the invention. Various changes may be made in the function and arrangement of elements.

Thus, various embodiments may omit, substitute, or add various procedures or components as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following systems, methods, and devices may individually or collectively be components of a larger system, wherein other procedures may take precedence over or otherwise modify their application.

The detailed description of exemplary embodiments herein makes reference to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

The embodiments described herein may also include methods described in the general context of computer-executable instructions, such as program modules, being executed by a processing device having specialized functionality and/or by computer-readable media on which such instructions or modules can be stored. Generally, program modules include routines, programs, objects, components, data structures, etc. that perform particular tasks or implement particular abstract data types. The embodiments described herein may also be practiced in distributed computing environments where tasks are performed by remote processing devices that are linked through a communications network. In a distributed computing environment, program modules may be located in both local and remote computer storage media including memory storage devices.

The embodiments described herein may include or be implemented in a variety of computer readable media. Computer readable media can be any available media that can be accessed by a computer and may include either or both of volatile and nonvolatile media, and similarly, removable, and non-removable media. By way of example, and not limitation, computer readable media may comprise computer storage media and communication media. Computer storage media include volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by a computer. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media. In some embodiments, portions of the described functionality may be implemented using storage devices, network devices, or special-purpose computer systems, in addition to or instead of being implemented using general-purpose computer systems. The term “computing device,” as used herein, refers to at least all these types of devices, and is not limited to these types of devices and can be used to implement or otherwise perform practical applications.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

As used herein, a process that is performed “automatically” may mean that the process is performed as a result of machine-executed instructions and does not, other than the establishment of user preferences, require manual effort.

As used herein, “wideband” is alternatively termed broadband, broad-spectrum, full-spectrum, or hearing bandwidth. Further, the term “acoustic immittance” is alternatively termed reflectance, acoustic admittance or the reciprocal, acoustic impedance.

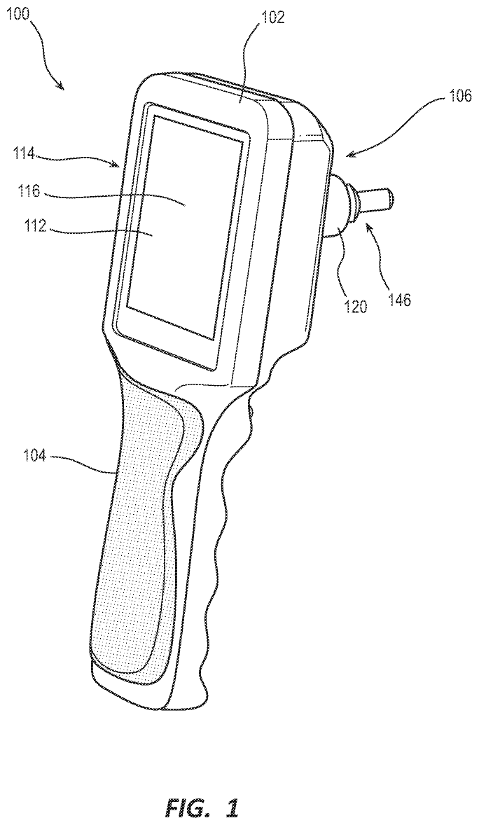

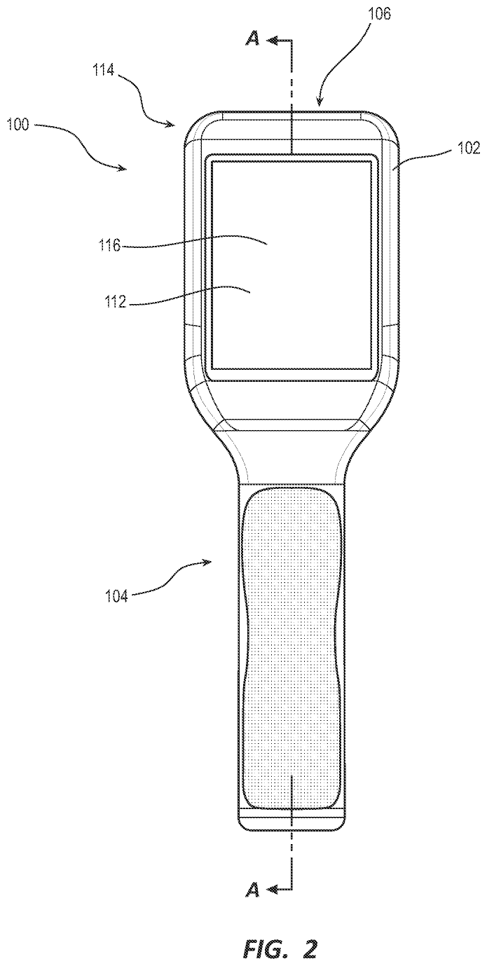

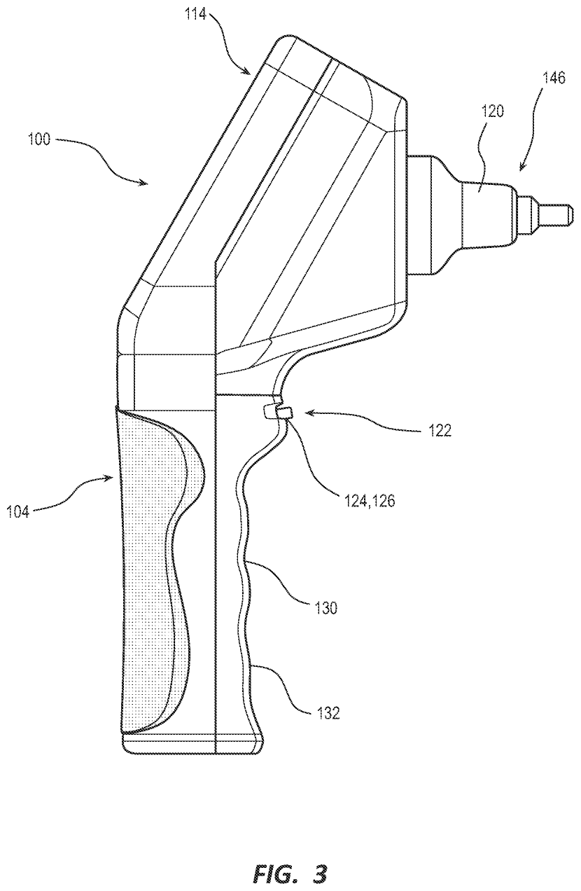

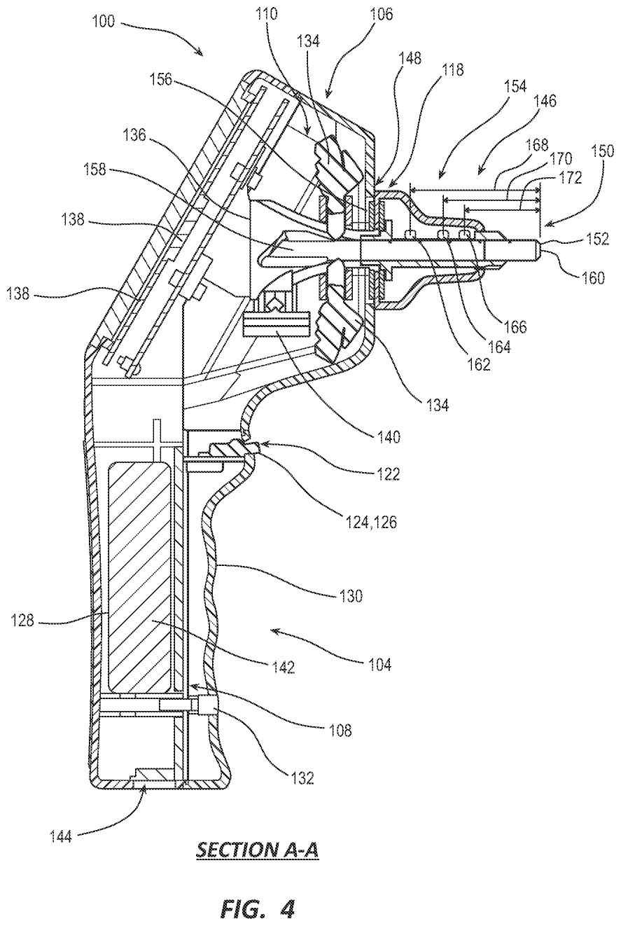

FIG. 1 illustrates a front view of an otoscope 100. FIG. 2 illustrates a front view of the otoscope 100. FIG. 3 illustrates a side view of the otoscope 100. FIG. 4 illustrates a side sectional view of a portion of the otoscope 100. Embodiments of the otoscope 100 may further include specific software methods for testing and evaluation of ears. The otoscope 100 may be used to conduct hearing tests and evaluations of infants, children, and adults. An embodiment of the otoscope 100 includes a digital video otoscope with integrated wideband acoustic immittance (WAI) and otoacoustic emission tests (OAE). The otoscope 100 delivers acoustic stimuli to the ear and measures acoustic responses using drivers and microphones located inside the device and entirely outside the ear canal. Accordingly, the otoscope 100 described herein enables a medical professional to conduct WAI and OAE testing without inserting invasive microphones in the ear and without the need to hermetically seal the ear canal with an acoustic probe. Although a hermetic seal can be used with the otoscope, if desired by the user or if helpful; for the test.

In one embodiment, the otoscope 100 may be configured to resemble a typically used otoscope. The otoscope 100 may include at least one speaker for sound input into an ear and a plurality of microphones to record sound waves input to the ear and sound waves received from the ear. An interface such as a track wheel enables a user to navigate one or more menus presented on the display screen 116. The otoscope 100 may also include a camera or lidar scanner for video streaming and/or still images of the eardrum in the visible spectrum and/or the infrared spectrum. The otoscope 100 delivers acoustic stimuli to the ear and measures acoustic responses using drivers and microphones located inside the otoscope 100, entirely outside the ear canal. Current WAI measurements and most OAE measurements require use of an insert driver and microphone hermetically sealed in the ear canal. The otoscope 100 avoids insertion of a driver and a microphone sealed in the ear canal by making equivalent measurements using the integrated, external microphones and speakers without sealing devices in the ear canal. Accordingly, the otoscope 100 described herein enables or user to conduct WAI and OAE testing without requiring skilled expertise of inserting invasive microphones in the ear and without the need for probe calibration using expensive equipment.

As shown in FIGS. 1-4, externally the otoscope 100 includes a shell 102 defining a handle 104 and an instrument portion 106 attached to the handle 104. The handle 104 defines a handle internal cavity 108 and the instrument portion 106 defines an instrument portion internal cavity 110. In the illustrated embodiment, the otoscope 100 includes a display device 112 partially positioned within the instrument portion internal cavity 110. Specifically, the instrument portion 106 defines a display opening 114 and the display device 112 is positioned within the display opening 114 such that a screen 116 of the display device 112 is positioned within the display opening 114 and is accessible by a user.

The instrument portion 106 further defines a tip opening 118 positioned on a side of the instrument portion 106 opposite the display opening 114. The tip opening 118 is configured to receive an interchangeable tip 120 that is configured to be inserted into the ear canal of a subject during an examination. As described herein, the tip 120 enables the otoscope 100 to deliver acoustic stimuli to the ear and measure acoustic responses. Additionally, the tip opening 118 enables the tip 120 to be interchangeable such that the tip 120 can be removed from the otoscope 100 and a new tip 120 inserted into the tip opening 118 such that a new sterile probe of suitable size may be used with each patient.

The handle 104 further defines an interface opening 122 configured to receive an interface 124 configured to enable a user to navigate the software of the otoscope 100. In the illustrated embodiment, the interface 124 includes a track wheel 126 that is rotated by the user to enable the user to navigate the software of the otoscope 100. Other suitable interface devices may be adapted for use in the invention. Additionally, the handle 104 may have an ergonomic design configured to conform to the shape of a user's hand when the user holds the handle 104. Specifically, the handle 104 has a first side 128 and a second side 130. The first side 128 has a rounded, cylindrical shape that conforms to the shape of a palm of the user's hand. The second side 130 is positioned opposite the first side 128 and defines a plurality of finger ridges 132 that conform to the shape of the fingers of the user's hand. The interface opening 122 and the track wheel 126 are positioned on second side 130 such that the user can use a finger to interface with the track wheel 126 to navigate the software of the otoscope 100.

Internally, the instrument portion 106 includes a plurality of speakers 134, an acoustic horn 136, at least one computing device 138, at least one camera 140, and a portion of the display device 112 positioned within the instrument portion internal cavity 110. As shown in FIG. 4, the speakers 134, the acoustic horn 136, and the camera and/or lidar 140 are positioned proximate the tip opening 118 such that the speakers 134, the acoustic horn 136, and the camera 140 may interface with the tip 120 to enable an examination of the ear canal. The speakers 134 and the acoustic horn 136 are configured to generate sound that is directed into the ear canal. The camera 140 is configured to generate video and/or images of the ear canal and the eardrum. The computing device 138 is configured to receive data from the speakers 134, the acoustic horn 136, the camera 140, the display device 112, the tip 120, and the track wheel 126 to analyze and display data on the display device 112.

The handle 104 includes a battery 142 and the track wheel 126 positioned within the handle internal cavity 108. In the illustrated embodiment, the battery 142 is a lithium ion battery that is rechargeable and provides power to the otoscope 100. The handle 104 defines a recharging jack 144 that enables the battery 142 to be recharged. As discussed above, the tracking wheel 126 is positioned on the second side 130 such that the user can use a finger to interface with the track wheel 126 to navigate the software of the otoscope 100. The tracking wheel 126 is partially positioned within the handle internal cavity 108 such that a portion of the track wheel 126 is exposed to the user's fingers and electronic components of the track wheel 126 are positioned within the handle internal cavity 108.

The tip 120 includes a sterile and replaceable ear speculum 146 that has a conical shape and defines a first opening 148 and a second opening 150. The first opening 148 is larger than the second opening 150 and is oriented towards the instrument portion 106 when the tip 120 is inserted into the tip opening 118. The second opening 150 is oriented away from the instrument portion 106 when the tip 120 is inserted into the tip opening 118. The tip 120 also includes a tube 152 extending from the first opening 148 to the second opening 150. Additionally, at least two microphones 154 are positioned on the tube 152 within the ear speculum 146. The tip 120 further includes an electronic interface 156 that interfaces with the computing device 138 to control the microphones 154 and to enable the tip 120 to be interchangeable.

As shown in FIG. 4, the tube 152 has a first end 158 and a second end 160. The first end 158 is positioned proximate the acoustic horn 136 and the second end 160 is positioned within the second opening 150 and is configured to be at least partially inserted into the ear canal. In the illustrated embodiment, the at least one microphone 154 includes a plurality of microphones 154. Specifically, in the illustrated embodiment, the plurality of microphones 154 includes three microphones 154, a first microphone 162, a second microphone 164, and a third microphone 166. In alternative embodiments, the tip 120 may include any number of microphones 154 that enable the otoscope 100 to operate as described herein including, without limitation, two, three, four, or more microphones 154. As shown in FIG. 4, the first microphone 162 is positioned a first distance 168 from the second end 160, the second microphone 164 is positioned a second distance 170 from the second end 160, and the third microphone 166 is positioned a third distance 172 from the second end 160.

The speakers 134 and the acoustic horn 136 are configured to generate planar sound or pressure waves that are directed through the tube 152 and the ear specula 146 into the ear canal. The ear generates an acoustic response to the forward sound wave traveling toward the TM and responds with a passive reflection and active otoacoustic emission traveling away from the TM. The microphones 154 are configured to receive and measure the pressure arising from both the forward and reverse components. The camera or lidar 140 is configured to generate video and/or images of the ear canal and the eardrum during the exam. Additionally, the tip 120 and/or the instrument portion 106 may include a light source (not shown) to aid the camera 140 and a lens (not shown) to enhance the camera. In the illustrated embodiment, the camera 140 includes a visible light camera. In alternative embodiments, the camera 140 may include any type of camera or lidar scanner that enables the otoscope 100 to operate as described herein including, without limitation, an infrared camera.

More specifically, as shown in FIG. 4, the speakers 134 are oriented toward each other and toward the acoustic horn 136. The orientation of the speakers enables them to generate a multifrequency, complex sound or pressure waveform that is planarized prior to entering the ear canal. More specifically, the geometry of the orientation of the speakers 134, the acoustic horn 136, and the tube 152 planarizes the complex pressure wave as it enters the ear canal. The orientation of the speakers 134 toward each other planarizes the complex sound or pressure wave as it is directed toward the ear canal. The complex sound or pressure wave becomes a substantially uniform pressure wave as the wave is planarized and enters and travels down the ear canal. The pressure wave traveling toward the TM is termed the stimulus herein. The complex sound or pressure wave stimulus partially reflects at the TM and comes back toward the otoscope 100 in the reverse direction along the ear canal. The reverse pressure wave consists of the passive reflection plus otoacoustic emissions generated inside the inner ear and traveling in the reverse direction toward the otoscope 100 at two or more different locations based on specifically spaced intervals that are known. The complex sound or pressure wave enters the tube 152 and is recorded by the microphones 154.

Specifically, the first microphone 162, the second microphone 164, and the third microphone 166 are each located at different distances 168-172 from the second end 160 of the tube 152 and, as such, each microphone 154 records the complex sound or pressure wave slightly differently. More specifically, in one embodiment the complex stimulus sound or pressure wave is constructed in the frequency domain from 30 sinusoids and is independently sent to the two speakers 134. The complex stimulus sound or pressure wave is generated at frequencies and multiple amplitudes. In another embodiment, the complex stimulus sound or pressure wave is generated at two or more closely spaced frequencies at a time to evoke acoustic responses from the ear at nonlinear distortion product frequencies. In another embodiment, the complex stimulus sound or pressure wave is a short duration click or chirp sound followed by quiet, which is a time domain stimulus. In one embodiment, the first microphone 162, the second microphone 164, and the third microphone 166 are each sampled at 96 kHz for 2 seconds and the data is analyzed in the frequency domain at the test frequencies and distortion product frequencies using discrete Fourier analysis. In another embodiment, digitized pressure data from the microphones 162 163 166 is analyzed in the time domain finite difference and integrations. In all embodiments, the analysis separately quantifies the forward sound wave traveling toward the TM (the stimulus) and the reverse sound wave traveling toward the otoscope 100. The computing device 138 is pre calibrated to analyze the complex sound or pressure wave.

During operations, a user selects a sterile tip to fit the patient's ear canal 120, attaches the tip 120 to an otoscope 100, and inserts the tip 120 into an ear canal of a patient. The speakers 134 generate a complex sound or pressure wave and the wave is directed into the ear canal of the patient. The wave is planarized as the wave enters the ear canal of the patient and the wave bounces off the tympanic membrane of the patient's ear. The wave travels back through the ear canal and into the tube 152 where the microphones 154 record the wave at different distances from the second end 160 of the tube 152. The stimulus wave is generated at different frequencies and times, and the otoscope analyzes the recorded wave and displays the physiological measurements/results to a medical professional or user. The user then uses the results to make decisions regarding health status of the patient's ear and hearing.

Accordingly, the otoscope 100 described herein enables a medical professional, nurse or technician to test a patient's hearing without the need for a hermetic seal or probe specific calibration. Specifically, the otoscope 100 according to an embodiment resembles an otoscope and includes at least one speaker for sound input into an ear and a plurality of microphones to record sound waves stimulus input to the ear and the otoacoustic response of the ear. The otoscope 100 delivers acoustic stimuli to the ear and measures acoustic responses using drivers and microphones located inside the otoscope 100, entirely outside the ear canal. Current WAI measurements and most OAE measurements require use of an insert driver and microphone sealed in the ear canal. The otoscope 100 avoids insertion of a driver and a microphone sealed in the ear canal by making equivalent measurements using the integrated, external microphones and speakers without sealing devices in the ear canal. Accordingly, the otoscope 100 described herein enables WAI and OAE testing without inserting invasive microphones in the ear and without requiring a hermetic seal and calibration by a trained professional.

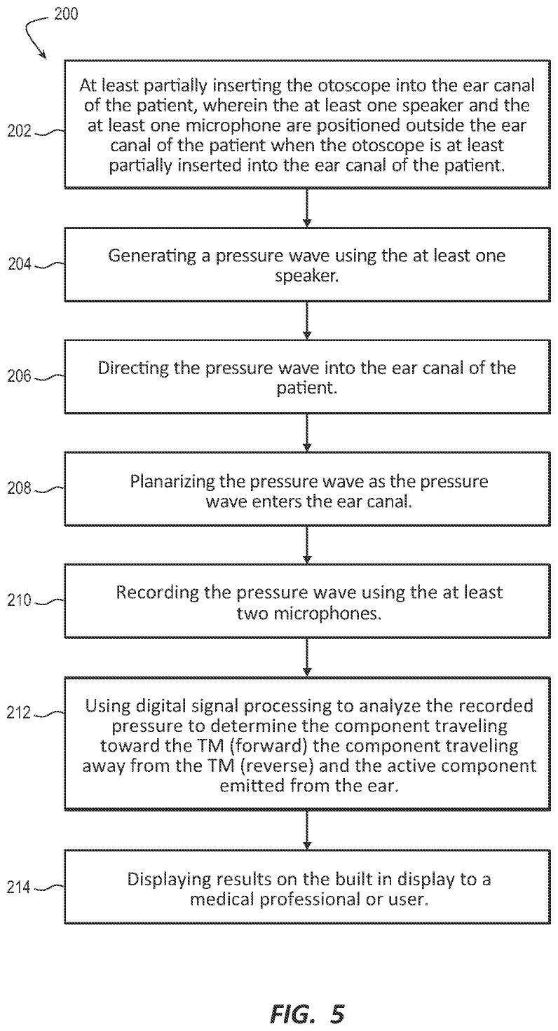

FIG. 5 illustrates a method 200 of examining an ear canal and a tympanic membrane of a patient using an otoscope. The otoscope includes at least one speaker and at least two microphones. The method 200 includes at least partially inserting 202 the otoscope into the ear canal of the patient. The at least one speaker and the at least one microphone are positioned outside the ear canal of the patient when the otoscope is at least partially inserted into the ear canal of the patient. The method 200 also includes generating 204 a pressure wave using the at least one speaker. The method further includes directing 206 the pressure wave into the ear canal of the patient. The method 200 also includes planarizing 208 the pressure wave as the pressure wave enters the ear canal. The method 200 further includes recording 210 the pressure wave using the at least two microphones. The method 200 also includes using 212 digital signal processing to analyze the recorded pressure to determine the component traveling toward the TM (forward) the component traveling away from the TM (reverse) and the active component emitted from the ear. The method 200 further includes displaying 214 results on the built in display to a medical professional or user.

The otoscope may include two speakers oriented toward each other, and wherein the method 200 may further include planarizing 214 the pressure wave as the pressure wave enters the ear canal. The otoscope may also include an ear speculum including a tube having a first end and a second end. The at least two microphones may include a plurality of microphones positioned on the tube at different distances from the second end of the tube. The method 200 may further include recording 216 the pressure wave using the at least two microphones comprising recording the pressure wave at different distances from the second end of the tube.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. An otoscope comprising:
at least one speaker or sound source configured to generate a pressure wave and direct the pressure wave toward an ear canal of a patient when the otoscope is at least partially inserted into the ear canal of the patient; and
at least one microphone configured to receive the pressure wave after the pressure wave has bounced off a tympanic membrane of the patient;
wherein the at least one speaker and the at least one microphone are positioned outside the ear canal of the patient when the otoscope is at least partially inserted into the ear canal of the patient; and
wherein the at least one microphone is configured to record the pressure wave, the otoscope is configured to analyze the pressure wave, and the otoscope is configured to display the results to a medical professional.

2. The otoscope of claim 1, comprising one or more speakers or sound sources.

3. The otoscope of claim 2, wherein the two speakers are oriented toward each other.

4. The otoscope of claim 2, wherein an acoustic horn planarizes sound from the source(s) to present a stimulus sound wave to the ear canal of the patient.

5. The otoscope of claim 4, wherein the pressure wave comprises a complex sound or pressure wave that is planarized as the complex sound or pressure wave enters the ear canal.

6. The otoscope of claim 5, further comprising an acoustic horn, wherein the sound sources and the acoustic horn are configured to generate the complex sound or pressure wave.

7. The otoscope of claim 6, wherein the two speakers are positioned on either side of the acoustic horn and are oriented toward each other and the acoustic horn.

8. The otoscope of claim 1, wherein the at least two microphones comprising a plurality of microphones.

9. The otoscope of claim 8, wherein the plurality of microphones comprising a first microphone, a second microphone, and a third microphone.

10. The otoscope of claim 9, further comprising an ear speculum and configured to be at least partially inserted into the ear canal of the patient.

11. The otoscope of claim 10, wherein the first microphone, the second microphone, and the third microphone are positioned within the ear speculum.

12. The otoscope of claim 11, wherein the ear speculum comprises a tube having a first end and a second end.

13. The otoscope of claim 12, wherein the first microphone, the second microphone, and the third microphone are attached to the tube.

14. The otoscope of claim 1, wherein the first microphone is positioned a first distance from the second end of the tube, the second microphone is positioned a second distance from the second end of the tube, and the third microphone is positioned a third distance from the second end of the tube.

15. The otoscope of claim 1, further comprising a camera configured to generate video and/or images of the ear canal and the eardrum.

16. The otoscope of claim 1, further comprising a lidar scanner configured to generate scans ear canal and the eardrum.

17. The otoscope of claim 15, wherein the camera comprises a visible light camera.

18. The otoscope of claim 15, wherein the camera comprises an infrared camera.

19. A method of examining an ear canal and a tympanic membrane of a patient using an otoscope, the otoscope includes at least one speaker and at least one microphone, the method comprising:
at least partially inserting the otoscope into the ear canal of the patient, wherein the at least one speaker and the at least one microphone are positioned outside the ear canal of the patient when the otoscope is at least partially inserted into the ear canal of the patient;
generating a stimulus pressure wave using the at least one speaker;
directing a planarized pressure wave into the ear canal of the patient;
recording the pressure wave using the at least one microphone; and
analyzing the recorded pressure wave to separate the forward component travel toward the TM from the reverse component traveling toward the otoscope.
displaying the results to a medical professional or user.

20. The method of claim 18, wherein the otoscope comprises two speakers oriented toward each other, and wherein the method further comprises planarizing the pressure wave as the pressure wave enters the ear canal.

21. The method of claim 18, wherein the otoscope comprises an ear specula including a tube having a first end and a second end, the at least one microphone comprises a plurality of microphones positioned on the tube at different distances from the second end of the tube, and wherein recording the pressure wave using the at least one microphone comprises recording the pressure wave at different distances from the second end of the tube.
